(19) United States
(12) Patent Application Publication
(10) Pub. No.: US 2002/0133502 A1
(43) Pub. Date: Sep. 19, 2002

(54) Method and system for interactive collection of information

(76) Inventors: Richard Nelson Rosenthal, New York, NY (US); Lewis Engel, Studio City, CA (US); Henry Beck, New York, NY (US); Larry Kenneth Davis, Richardson, TX (US); Justin Dale Schaper, Plano, TX (US)

Correspondence Address: BROWN, RAYSMAN, MILLSTEIN, FELDER & STEINER LLP, 900 THIRD AVENUE, NEW YORK, NY, 10022, US

(21) Appl. No.: 09/755,973

(22) Filed: Jan. 5, 2001

(51) Int. Cl. G06F 7/00; A61B 19/00; G06F 17/00

(52) U.S. Cl. 707/104.1; 706/924; 128/920

(57) ABSTRACT

A method of collecting participant replies into a database residing on computing devices. The participant replies provide information of particular interest about the participant. The replies are provided in response to questions selected by an overseer. The questions are grouped into question sets; each question set addressing a particular aspect of information being sought. The question sets are further grouped into lists of the question sets determined by an overseer in accordance with the participant replies, wherein each participant is assigned a list. The invention establishes data paths over communication networks for communicating the questions and replies. For each participant a next question in the list is determined based on the participant's previous replies. The determined next question is communicated to the participant via the data path and the replies are communicated back from the participant via the data path. After all the questions on the list are asked a report is created and forwarded to the overseer who may change the make up of the list of question sets for the participant.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

This invention relates to collecting information in response to pre-defined questions with the aid of a computer and more specifically to using the response information to pose additional subsequent dynamically selected pre-defined questions.

The advent of managed health care has resulted in enormous pressure to cut costs in the delivery and management of medical treatment. According to studies by Mamlin, Mamlin, J. J. and Baker, D. H., Combined Time-Motion and Work Sampling Study in a General Medicine Clinic, Medical Care 11:449-456, 1973 and Korpman, Korpman, R. A. and Lincoln, T. L., The Computer-Stored Medical Record: For Whom?, Journal of the American Medical Association, 259:3454-3456, physicians spend an estimated 38% of their time writing patient records; nurses spend 50% of their time doing so. An estimated 35%-39% of total hospital operating costs have been associated with patient and professional paperwork, Richart, R. H., Evaluation of a Medical Data System, Computers and Biomedical Research, 3:415-425. As a result, perhaps 20% by conservative estimates, or $200 billion a year of current health care costs are spent on capturing, documenting, and distributing information (largely on paper or in “hard copy”) among providers of medical services, payers and others, Information Infrastructure for Healthcare, (95-10), a booklet about the Advanced Technology Program of the National Institute of Standards and Technology (1995: NIST, Department of Commerce, Gaithersburg, Md. 10899-0001), p. 9.

A study of doctors done at Boston University in 1994 found that 90% of all doctor-patient interaction in a clinic could have taken place remotely via telecommunications without the doctor and patient meeting in person, Cited in the Boston Sunday Herald, Oct. 8, 1995, source: Dr. Paul Gertman. In another study, Wasson, J., Gaudette, C., Whaley, F., Sauvigne, A., Baribeau, P., Welch, G., Telephone Care As a Substitute for Routine Clinic Follow-Up, Journal of the American Medical Association, 1992; 267(13): 1788-1793, substitution of clinician-initiated telephone calls for some clinic visits reduced medical care utilization without adversely affecting health status. Telephone-care patients had significantly fewer total clinic visits, less medication use, fewer days of hospitalization, and fewer intensive care unit days than patients assigned to in-person care. Estimated total care expenditures for the telephone-care group were 28% less per patient.

Home Care

Over seven million people received home care visits in 1995, Basic Statistics About Home Care 1995, a Publication of the National Association for Home Care, page 1. Since it is common for persons to receive such visits so long as their conditions warrant or their insurance permits, each such person received an average of 61 home care visits in 1994. That is to say, there were a total of more than 429 million home care visits in the U.S. in 1994, and undoubtedly 15 more visits took place in 1995, Schiller, Arthur E., Jr., The CPR [i.e., computer-based patient record]: A Patient/Consumer Perspective, draft MS. submitted to Health Informatics, March 1996 issue, page 3.

A frequent problem with chronically ill patients being treated at home is that they often wait too long to seek treatment for developing medical problems or fail to adhere to a prescribed 20 treatment regimen until they have become quite ill. Such situations often mean that such patients frequently must be readmitted to emergency rooms and hospitals once their conditions have been reported, a large and often avoidable expense. Any technology that could reduce the incidence of such problems would be of great benefit to insurers and home care patients alike.

In addition, any rationalization of current systems which could reduce the number of 25 home care visits generally without reducing quality of care could also give rise to large cost-savings, simply because of the sheer cost of such visits. Finally, any technology which could improve the likelihood that persons receiving health care took their medication as directed or otherwise followed a prescribed treatment regimen would also result in great savings, since the failure to comply with prescribed treatment regimens is a common cause of treatment “failure.”

Data Interchange

The application of Electronic Data Interchange (“EDI”) technology to medical claims processing has been a long-standing goal of insurance companies, health care maintenance organizations and other large payers. Such systems, as traditionally conceived, transmit information stored in the computers of those seeking payment (usually providers of health care services under contract to the payer-insurer) directly into the computers of the payer. Hence, “electronic data interchange.”

However, despite the evident cost and other benefits of such systems, their adoption has been slow. One reason has been that providers of health care services have often refused to take part in collaborative electronic data interface (EDI) projects with their payers. They have, for example, been disinclined to spend the substantial amounts of money required to create the necessary compatibility between their systems and those of their payers. They have been wary of establishing a direct connection between their systems and those of their payers for fear of compromising the security of their own patient records. And the payers, in turn, have been unwilling for reasons of cost to bear the entire cost of retrofitting their providers' computer systems so as to make them compatible because to do so would likely be more expensive than the current, though cumbersome and expensive, hard copy and mail system.

As a result, over 80% of medical claims submitted are still submitted in hard copy by mail. This slows the claims fulfillment process, and creates hundreds of millions of dollars of unnecessary overhead and processing costs at payer organizations and accounts receivable financing costs on the part of health care providers.

Telemedicine

Interactive computer telephony applications are in use in a growing number of industries. Such applications may for example be encountered when purchasing movie tickets by phone. More sophisticated versions include accessing a brokerage or bank account telephonically without the intervention of human intermediaries. All of these computer telephony applications, it should be noted, ride “piggyback” on the often taken-for-granted worldwide telecommunications infrastructure or telephone network. It is this infrastructure that provides much of the leverage for the computer telephony applications.

There has been some disclosure of interactive computer telephony applications in the field of health care.

Examples of other activity in the field of telemedicine include the transmission of x-ray images over phone lines and teleconferencing for consultation purposes among geographically separate medical centers.

For example, U.S. Pat. Nos. 4,792,968, 4,845,739, 4,975,945 and 5,349,633 disclose call processing systems using voice units, and the following disclose the use of telephone and/or computers for information collecting and/or monitoring in the health field: U.S. Pat. Nos. 5,633,910 and 6,014,626; Mundt, James C., et al., Administration of the Hamilton Depression Rating Scale Using Interactive Voice Response Technology; Kobak, Kenneth A., et al., Computerized Assessment in Clinical Drug Trials; Greist, John H., et al., Telephone Assessment Program: Patient Monitoring and Clinician Feedback; Mundt, James C., et al., An Application of Interactive Voice Response (IVR) Technoloy to Longitudinal Studies of Daily Behavior; Kobak, Kenneth A., et al., Computer-Administered Clinical Rating Scales—A Review; Greist, John A., et al., The Telephone Assessment Program: Efficient Patient Monitoring and Clinician Feedback; Mundt, James C., et al., Cyles of Alcohol Dependence: Frequency-Domain Analyses of Daily Drinking Logs for Matched Alcohol-Dependent and Nondependent Subjects; Perrine, and Ph.D., M. W., et al., Validation of Daily Self-Reported Alcohol Consumption Using Interactive Voice Response (IVR) Technology.

There is a need, however, to improve the process for collecting and using information, particularly in the health care field, and to reduce the time spent by personnel in collecting such information and in inputting, processing, using, etc. such information.

### SUMMARY OF THE INVENTION

It is an object of the present invention to improve telemedicine. It is another object to do so in a simple, appropriate and cost-effective manner. Another object is to implement applications of telemedicine with low-cost, easily available technology, which rides “piggyback” on the existing telecommunications infrastructure.

It is another object of the present invention to dynamically gather information without duplication and with sensitivity to replies as well as to related previously supplied information.

It is a further object of the present invention to automatically adjust the scope of the information being gathered regarding individual participants.

It is yet another object of the present invention to compile a knowledge base, which may be used to provide a historical basis for automatically adjusting the scope of the information being gathered from separate participants.

The invention achieves the above and other objects and provides for computerized data collection from persons with the participation or intervention of overseers, and/or dynamic question selection for presentation to a respective person.

A respective overseer participates in the collection of information from a respective person. The overseer may participate in the selection of initial questions, or in the selection of subsequent questions based on answers to initial questions, or both.

The term “overseer” is used in a broad sense and encompasses anyone involved with preliminary questioning or screening or more detailed questioning, or anyone involved with administration of any program in which a person questioned may be involved, as well as a physician or physiologist where the program is a course of diagnosis and/or treatment of a patient. In addition, the term “overseer” may encompass more than one person, or different persons at different times during the coarse of obtaining information from a person.

Questions are stored in a database, and any question can be accessed from the database and presented to a person in dependence upon one or more answers to questions.

In a preferred embodiment, questions are arranged into questions sets, and lists of questions sets are constructed for presentation to respective persons. Presentation of questions sets in a list and individual questions in a question set is determined in dependence upon answers received to one or more prior questions. An initial list (for a person who has not been presented with questions before) and subsequent lists may be constructed with the participation of an overseer.

The initial list may be the same for all persons or groups of persons. Presentation of questions sets in an initial list and individual questions in question sets is determined in dependence upon received answers. Alternatively, an initial list may be constructed with the participation of an overseer.

Questions may be selected on a dynamic basis based on the answers to one or more prior questions. Branching for navigating to at least some questions need not be determined simply by coding, and theoretically, any question in the database may be selected based on processing of one or more answers to prior questions.

A method is disclosed of collecting participant replies into a database residing on computing devices. The participant is able to access a computing device via a data path over a telecommunications network or over a digital network, e.g., the Internet. Data paths are established over the networks for communicating questions, replies, and reports. The participant replies provide information of particular interest either within the scope of the participant's knowledge or about the participant. As mentioned, questions are selected with the participation of an overseer.

The questions are stored in a questions database and are created in advance (e.g., well in advance) of the information collection or they may be created while the information collection is taking place. Adjustment and modification of questions may also be achieved while the information collection is taking place. The questions are grouped into question sets, each question set addressing a particular aspect of the information being sought. For each participant the question sets are grouped into lists of question sets. As mentioned, an overseer in accordance with the participant replies determines these lists.

For each participant, after the participant has replied to a branch question, a next question in a question set of a list is determined based on the participant's previous replies. (Here “branch” is used in a broad sense not dependent upon how the branch is implemented or the next question selected.) The determined next question is communicated to the participant via the data path and the replies from the participant are communicated back to the computing device via the data path. After questions from questions sets of a list have been presented and answers 1 o received, the current list my be adjusted by an overseer. Alternatively, after questions on all questions sets in a list have been asked, a report is created and is forwarded to the overseer who then may change the make up of a next list of question sets for the individual participant, after which the collection of information may continue.

A method according to the invention obtains information from a person using a computer with the participation of an overseer. The method includes the steps of providing at least one first (e.g., initial) set of questions to be presented to the person. The computer generates questions from within each first set which are presented to the person. The computer receives answers from the person to the questions presented from the at least one second set of questions; each question presented to the person from the at least one first set being selected by the computer from the at least one first set of questions independently of answers received from the person or in dependence upon at least one answer received from the person; providing at least one second set of questions to be presented to the person in dependence upon at least one answer received to at least question presented from at least one first set of questions, the at least one second set of questions being selected through intervention by the overseer; computer generating questions from the at least one second set of questions and presenting those computer generated questions to the person; and receiving in the computer answers from the person to questions presented from the at least one second set of questions.

The computer may select the first question set or sets with or without overseer participation. However, an overseer participates in the selection of at least one second set of questions. As mentioned, preferably lists of question sets are constructed. For example, a first or initial list of first questions may be constructed, and a second or subsequent list of second questions may be selected.

Subsequent sets of questions or lists may pose questions tracking a persons participation in a particular program.

In a preferred embodiment, a first or initial set or questions or list of question sets may implement a screener function, while subsequent question sets or lists of questions sets may implement exploring and/or tracking functions.

Second sets of questions, and second or subsequent lists of question sets may be determined and presented a plurality of times. In other words, information collection may continue after a second set of questions or second list of questions sets have been presented, each subsequent set or list being referred to generally as a second set or list.

A method of the invention for obtaining information from a person using a computer comprises the steps of: providing at least one set of questions to be presented to the person from a database of question sets; retrieving from the database questions from the first set and presenting those questions to the person; and receiving in the computer answers to questions presented to the person. At least one question is retrieved from the database in dependence upon an answer to an earlier presented question in a dynamic fashion, i.e., not as a result of a hard coded branch but dynamically where any question in the database is eligible for selection.

A system of the invention for obtaining information from a person comprises a computer, a computer readable memory storing questions and in association with the person answers to the questions presented to the person, the memory being accessed by the computer, and programming. The computer is programmed to select any question of a plurality of questions stored in the memory to present to a person in dependence upon any answer of a plurality of answers stored in the memory.

### BRIEF DESCRIPTION OF THE DRAWINGS

The foregoing objects and advantages of the present invention may be more readily understood by one skilled in the art with reference being had to the following detailed description of a preferred embodiment thereof, taken in conjunction with the accompanying drawings wherein like elements are designated by identical reference numerals throughout the several views, and in which:

FIG. 1a is a network topology diagram showing connectivity of components of the present invention.

FIG. 1b is a diagram showing component parts of the present invention.

FIG. 2 is a diagram showing connectivity of component parts comprising the computing devices utilized by the present invention.

FIG. 3a is a record layout diagram showing record fields comprised in each record of a questions database of the present invention.

FIG. 3b (1-4) are a compilation of representative records showing a sample of actual data comprised in each record of the questions database of the present invention.

FIG. 3c (1-4) are logical flow or branching diagrams describing the sequences followed in the sample of questions shown in FIG. 3b (1-4) when these questions are posed and replies are received by the inventive system.

FIG. 3d is an alternative record layout diagram (to that of FIG. 3a) showing record fields comprised in each record of the questions database of the present invention.

FIG. 4 is a record layout diagram showing record fields and actual data comprised in records of the answers database of the present invention.

FIG. 5 is a flow diagram showing a sequence of steps from initiation of contact between participants, the overseer and the system of the present invention.

FIGS. 6a and 6b are flow diagrams showing a sequence of steps followed by the explorer process of the present invention.

FIGS. 7a -7c are sample reports showing analyzed reply data of the present invention.

### DETAILED DESCRIPTION OF A PREFERRED EMBODIMENT OF THE INVENTION

Developments in the electronic computing field in general and computer-telephony in particular make it possible to capture, organize and analyze large amounts of health-care relevant information. Such information gathering may be achieved reliably and at low cost using standard computing devices, analog, touch tone, and digital telephones, fax machines and the existing Internet and telecommunications infrastructure linked to existing informational resources. This, in turn, permits the reengineering of workflow settings in which vast amounts of critical information must be managed, and the replacement in many instances of administrative and clerical tasks that are presently performed by highly paid personnel with tasks performed by inexpensive technology.

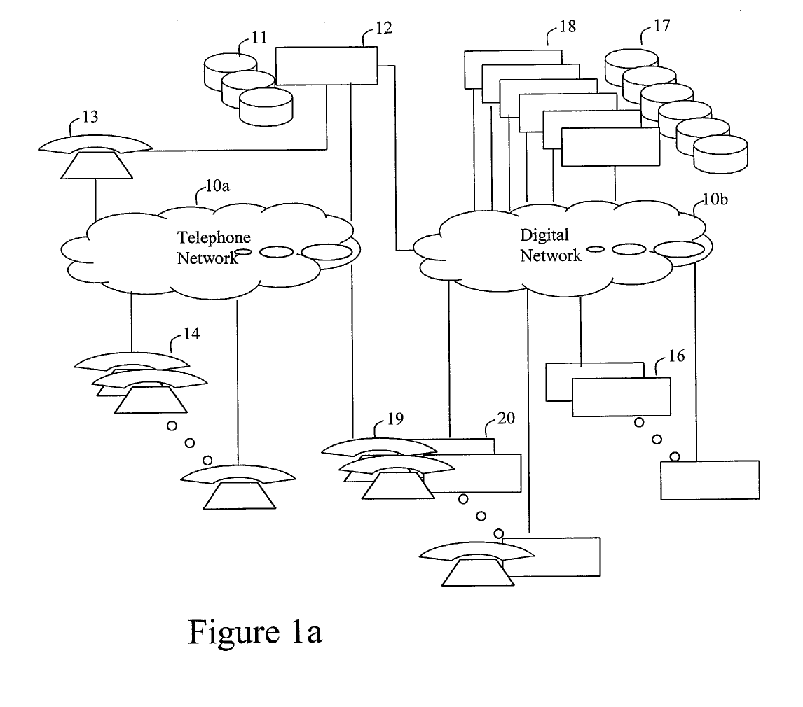

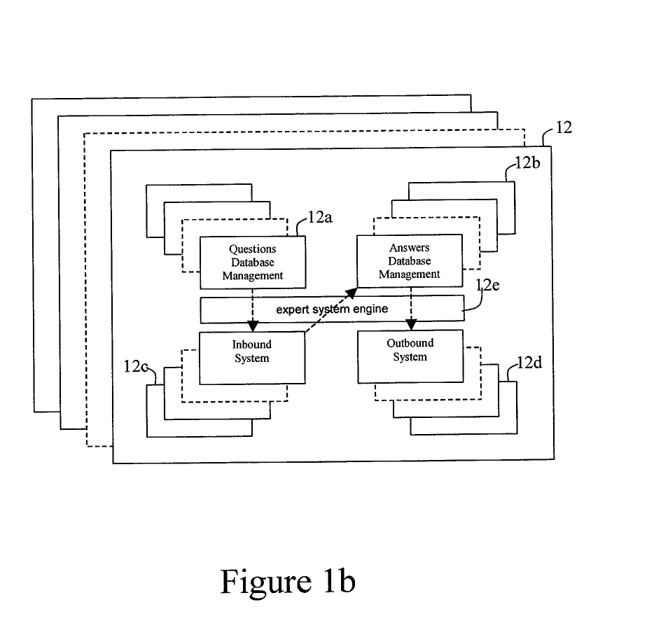

The present invention may be implemented over a plurality of communication networks. In one preferred embodiment, as shown in FIG. 1a, the present invention uses a digital computer network 10b such as the Internet and a telephone network 10a. Alternative embodiments of this invention may use any one of these networks separately; other embodiments may use other additional networks.

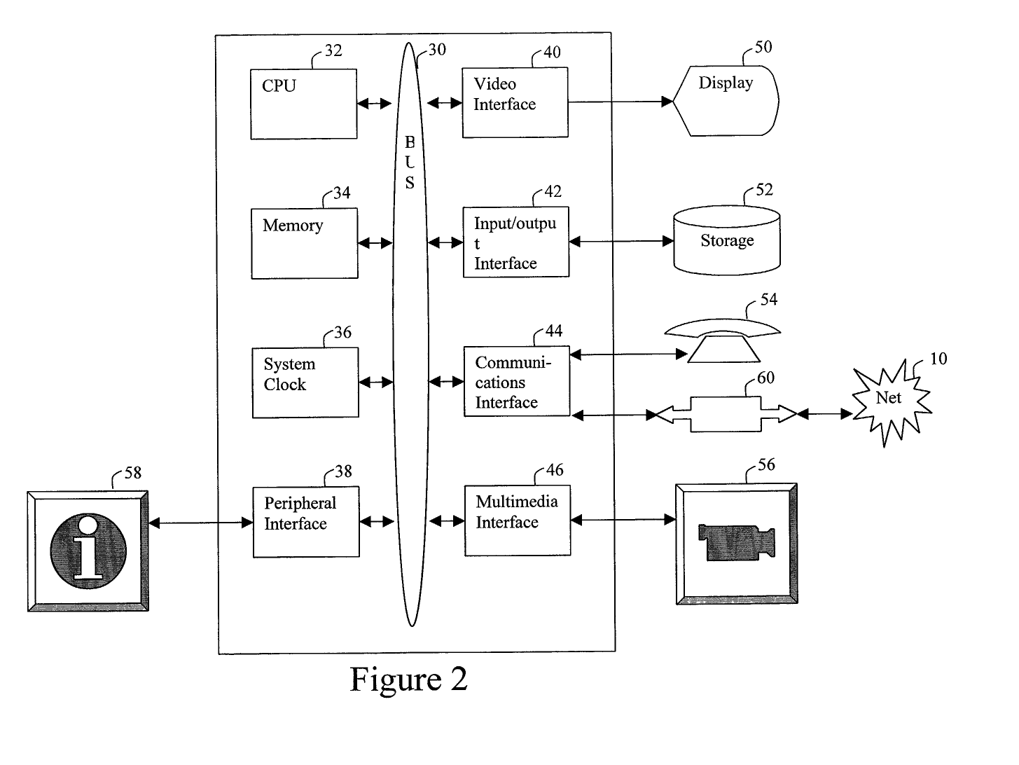

Computing devices 12, 16, 18, and 20 may illustratively take the configuration of any computer ranging from a mainframe to a personal computer (PC) to a hand-held computing device. In one illustrative embodiment of this invention as shown in FIG. 2, such computing devices may comprise a bus 30, which is connected directly to each of the following: a central processing unit (CPU) 32; a memory 34; a system clock 36; a peripheral interface 38; a video interface 40; an input/output (I/O) interface 42; a communications interface 44; and a multimedia interface 46. The common bus 30 is further connected via the video interface 40 to a display 50; via the I/O interface 42 to a storage device 52, which may illustratively take the form of memory gates, disks, diskettes, compact disks (CD), digital video disks (DVD), etc.; via the multimedia interface 46 to a multimedia component 56; via a peripheral interface 38 to one or more peripherals 58, such as a keyboard, a mouse, navigational buttons, e.g., on a digital phone, a touch screen, and/or a writing screen on full size and hand held devices, e.g., a Palm Pilot™; via the communications interface 44, e.g., a plurality of modems, to a network connection 60, e.g., an Internet Service Provider (ISP), and to other services, which in turn are connected to the network 10, whereby a data path is provided between the network 10 and the computing device and, in particular, the common bus 30 of these computing devices; and furthermore, via the communications interface 44 to a wired and/or a wireless telephone system 54.

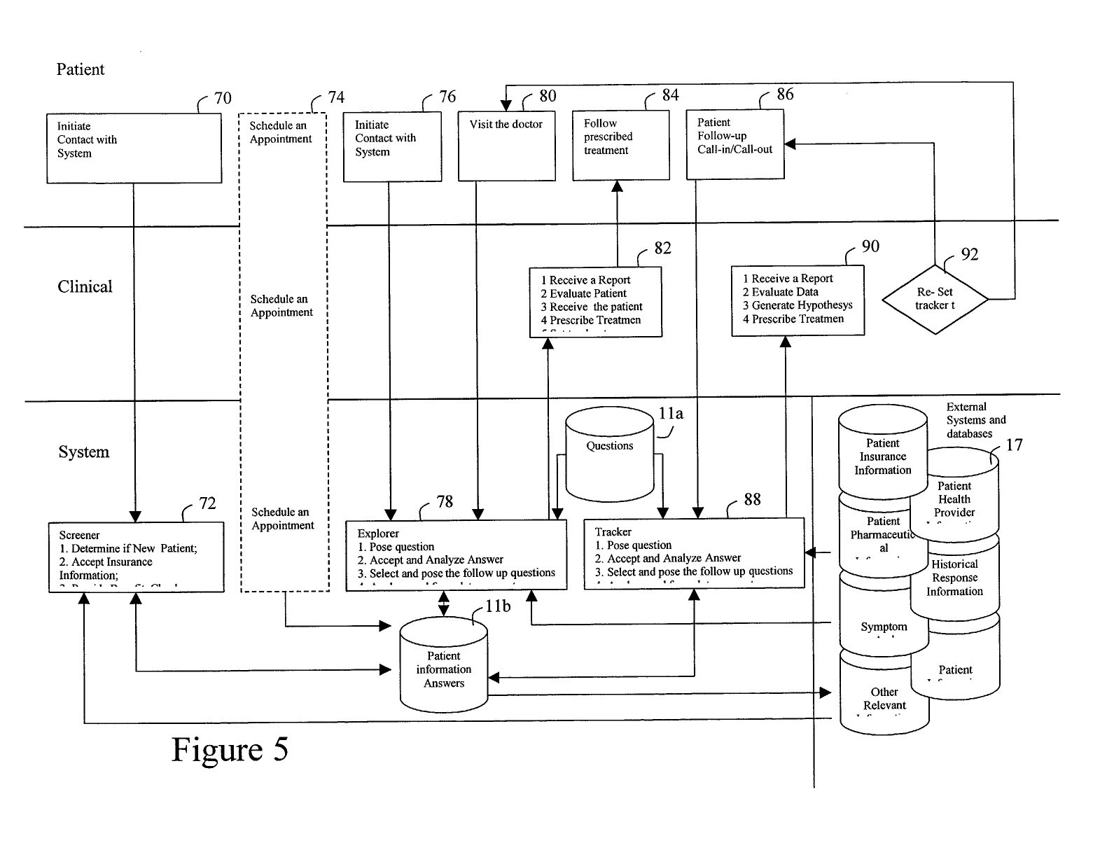

The computing device 12 comprises inventive telephony and database management applications, shown in FIG. 1b. It is appreciated that one skilled in the art could have used a single computing device 12 or multiple computing devices 12 to implement the inventive telephony and database management applications without departing from the scope of the invention. The telephony applications comprise one or more inbound and outbound systems 12c and 12d, which may but do not have to be resident on the same computing device 12. If multiple iterations of these systems are initiated each iteration may be executed on a separate computing device 12 or on the computing device 12 shared with any combination of other component systems 12a-12d. Additionally, an expert engine system 12e is utilized in a manner described below in conjunction with FIGS. 5-7.

The inbound systems 12c may comprise applications such as the Call Center used to receiving inbound calls via the telephone network 10a (FIG. 1a) and for performing tasks to intelligently route these calls to the appropriate receivers 14 (FIG. 1a). The inbound calls may convey information through the Interactive Voice Response (IVR) systems that use the keypad on the telephone receivers as a data entry mechanism. The inbound information may alternatively be conveyed to the computing device 12 (FIG. 1a) through the use of common web browser programs such as Microsoft® Explorer and Netscape® Navigator. Such programs may reside on the appropriate computing devices 16 (FIG. 1a) and interact with the Inbound Systems 12c. Messaging applications using any store-and-forward schemes, e.g., voice-mail, fax-mail, and e-mail may also be used.

The outbound systems 12d may comprise at a minimum, a single user telephone handset application operating via a computer. Such an application may be expandable to a plurality of predictive dialing applications, where the computer generates many outbound calls. The outbound systems 12d may further comprise the Interactive Fax applications, which include networked (1) fax servers, where a fax machine becomes a network node and sends outbound server files; (2) fax broadcasting, where the same fax is sent to a list of recipients, often simultaneously, using several phone lines; or (3) fax on demand, where callers dial into a system and request that documents be sent to them.

The database management applications comprise questions and answers database management systems 12a and 12b. It is appreciated that one skilled in the art can manage a single database 11 (FIG. 1a) or multiple databases on a single computing device 12 or multiple computing devices 12 to implement the inventive database management applications without departing from the scope of the invention. The structure of databases 11 will be described below.

The questions database management system 12a acts as a “paperclip,” i.e., a mechanism connecting varying number of components, to accommodate scalability in the following circumstances.

Horizontal

1) Other component systems 12b-12e may be expanded as needed, i.e., additional iterations of component systems 12b-12e may be started up while the database management system 12aremains static, i.e., only one iteration is launched to manage a database comprising particular questions, e.g., medical. The need to grow such component systems 12b-12e will arise, for example when the capacity of one iteration of the inbound system 12c is exceeded and additional iterations of the inbound system 12c may be required to accommodate more users.

Vertical

2) Additional iterations of all component systems 12a-12e are started up, in order to accommodate different applications launched to manage multiple diverse databases 11a(FIG. 1a) or the same questions for different clients.

Horizontal and Vertical

3) Additional iterations of the database management system 12a may be started up while other component systems 12b-12e (i) remain static or (ii) follow the horizontal scalability model described above. This is achieved in order to accommodate different applications launched to manage multiple diverse databases 11a (FIG. 1a) comprising questions, e.g., for medical applications, commerce applications, academic applications, while sharing the same component systems 12b-12e.

Referring back to FIG. 1a, the databases 11 include the questions to be posed to participants and the replies given by the participants to such posed questions, including the participants' personal information, e.g., voice print files for authenticating the participant. Depending on the particular use to which the inventive system is put or its utility, the participants may include medical and psychiatric patients, homebound patients and the elderly, subjects of studies and surveys, consumers in general, students, and others.

The inbound system 12c (FIG. 1b) component of the inventive system collects information from the participants. The information is in the form of the replies given in response to the questions posed by the system. To receive the participants' replies, data paths are established between the inbound system 12c component and (1) the participants using telephonic devices 14, such as telephones or fax machines, via the network 10a; (2) the participant using the computing devices 16 via the network 10b, the computing devices 16 can be set up as an Internet website.

A separate data path via the network 10b may further be established to the external computing devices or websites 18. These websites 18 manage or maintain databases 17 that include information regarding the participants. In our illustrative embodiment describing the medical application of the present invention, such information may include and identify the participant's insurance, health care provider, health maintenance organization (HMO), medications taken, e.g., pharmacy records, referring physician, as well as tables on illness, symptoms, etc.

After the questions have been posed, the replies to these questions are collected via the inbound data paths described above. This may be achieved through computer enabled IVR, e-mail, Internet website posting, interactively through the Internet browser programs, and by scanning handwritten replies to printed or mailed questionnaires. These questionnaires may be received at the inbound system 12c (FIG. 1b) through regular mail and/or facsimile equipment 13 connected to the computing device 12.

The outbound system 12d (FIG. 1b) analyzes the replies received from the participants along with information relevant to the particular participant it retrieves from databases 17. The reports are created using the information, and are then sent to an overseer, for example, a treating physician, a teacher, or a merchant, via the networks 10. These reports may be sent as electronic transmissions to the computing devices 20 and/or telephonic devices 19, e.g., fax machines. If the report is sent as an e-mail message, the overseer may use the computing device 16 to retrieve it from some e-mail server on the Internet.

“Questions” Database

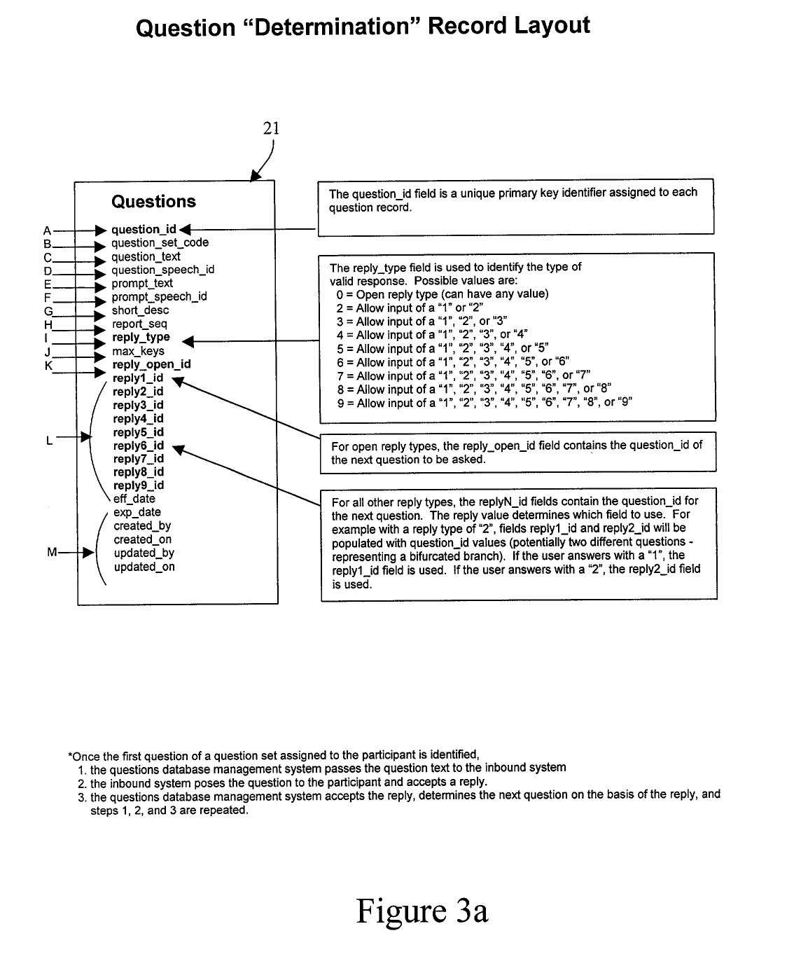

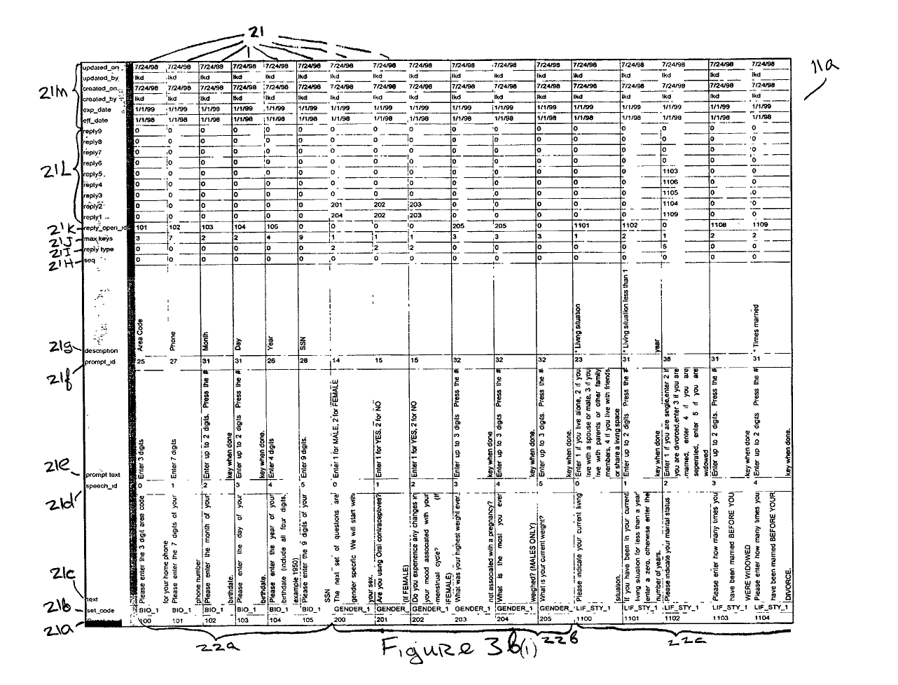

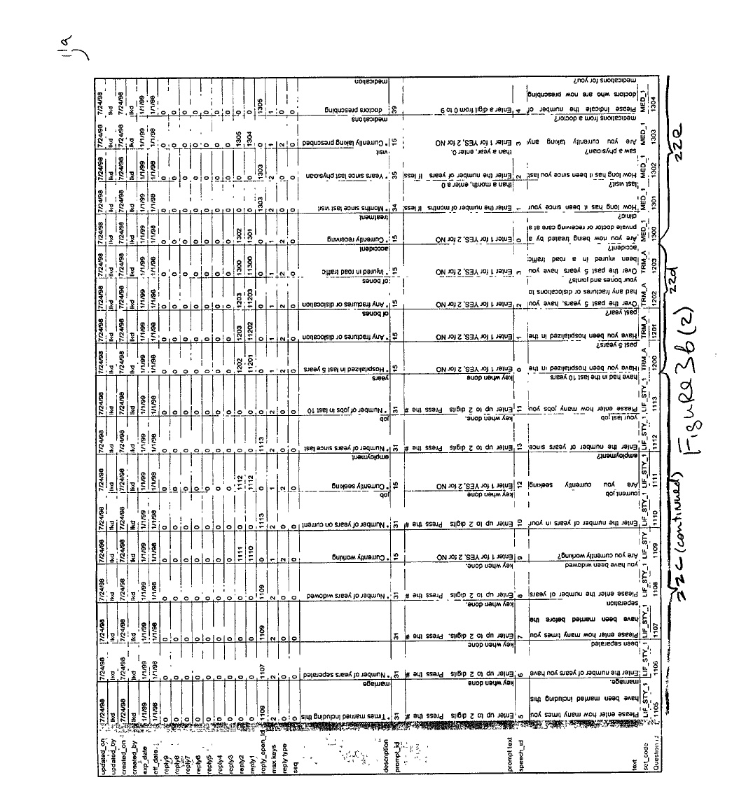

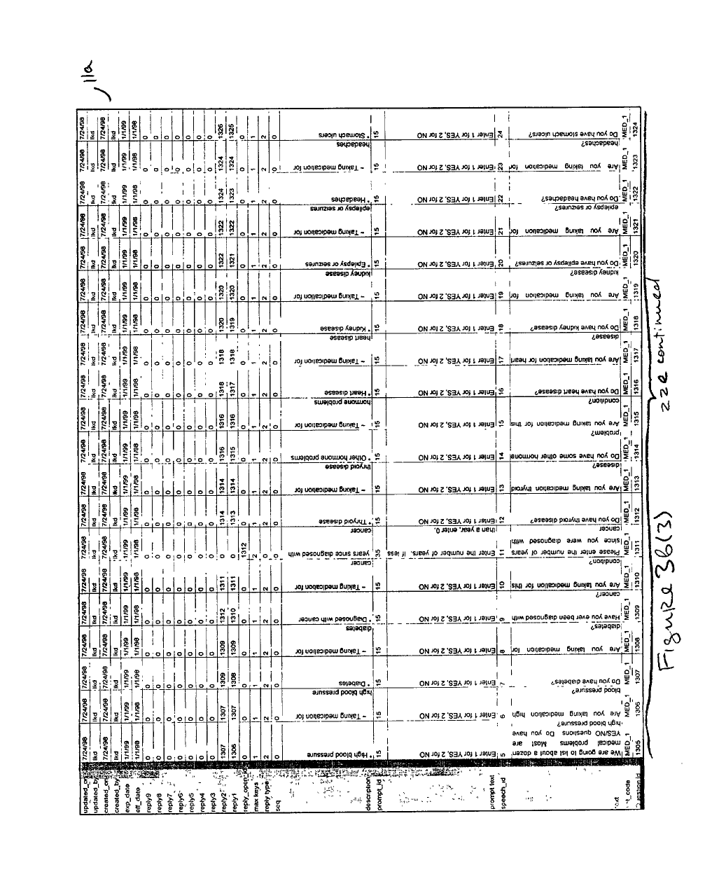

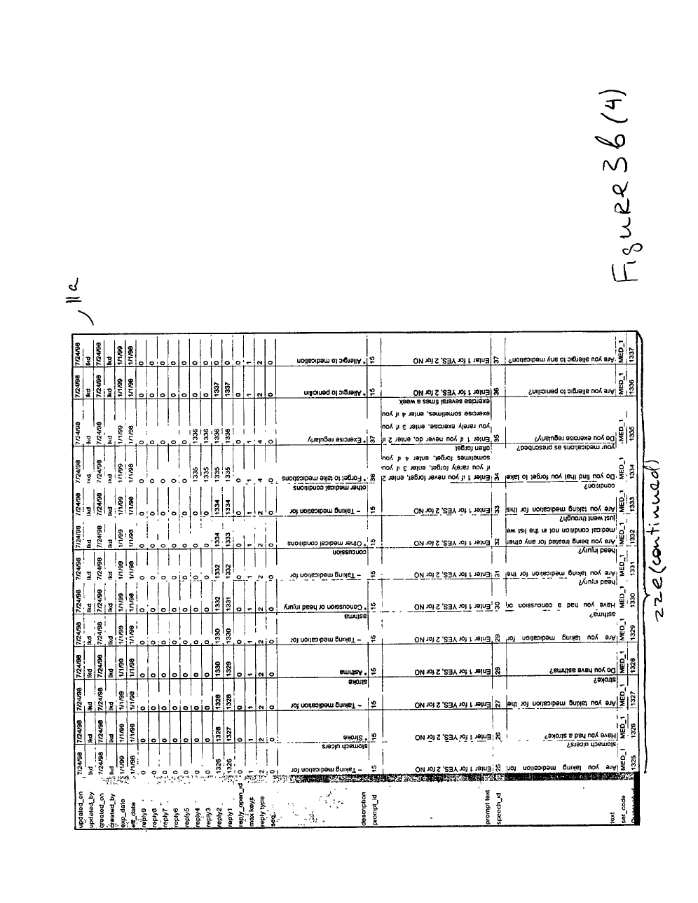

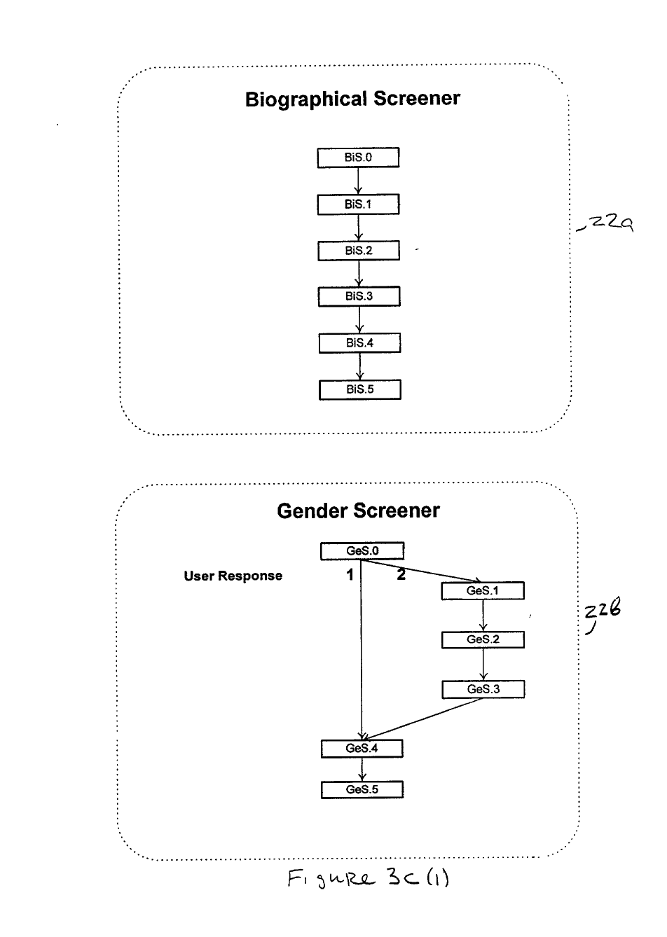

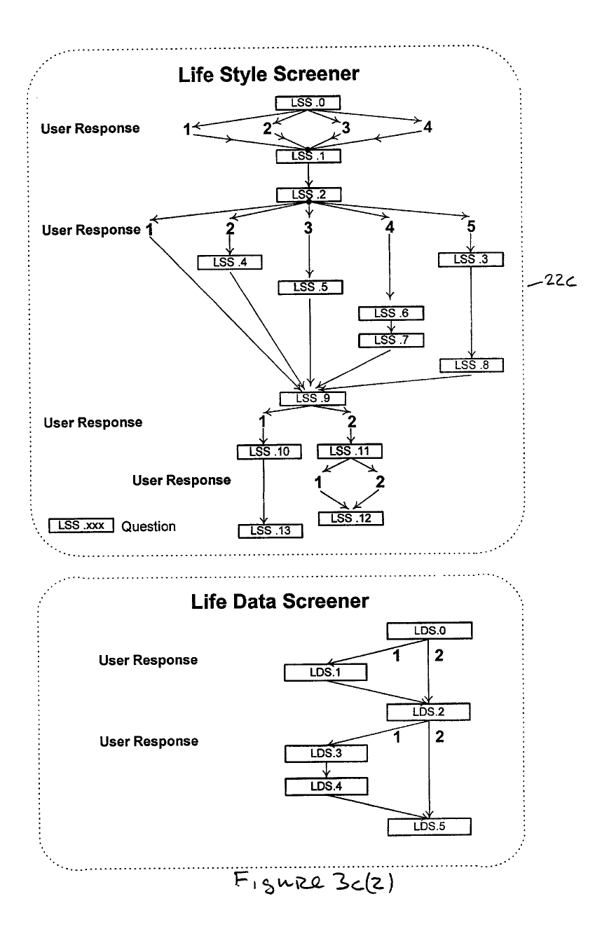

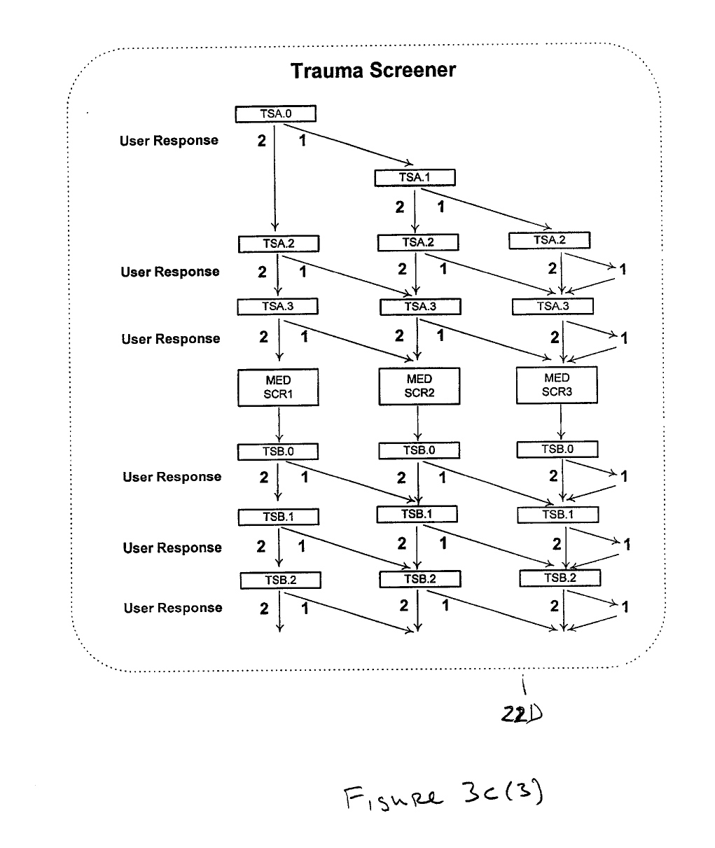

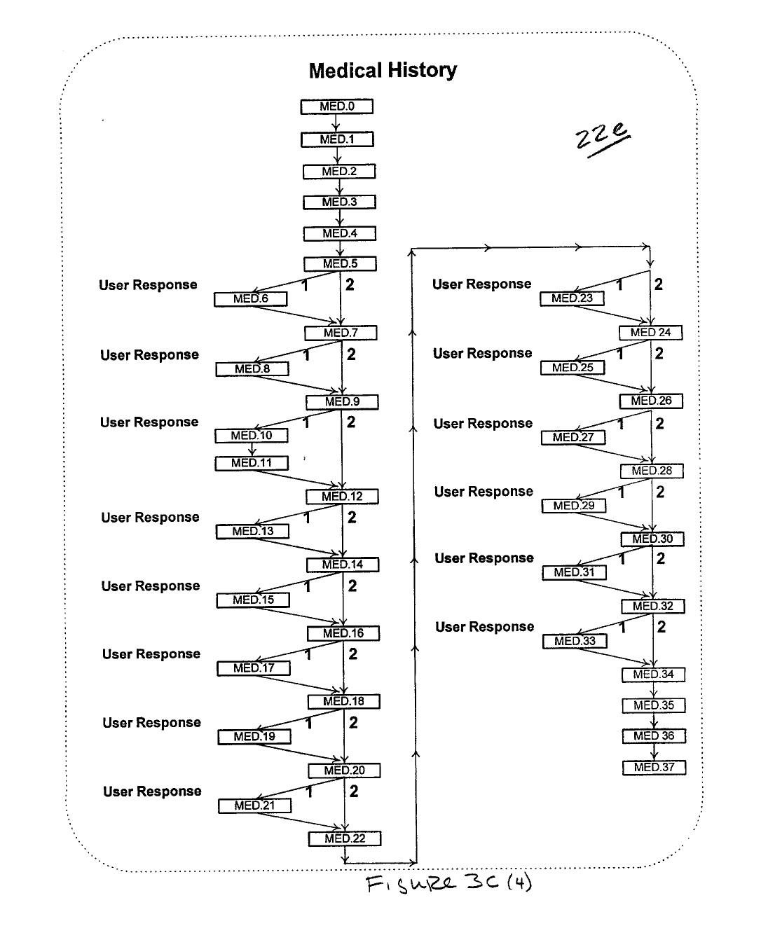

The record layout and an example of the questions database 11a is shown in FIGS. 3aand 3b, respectively. In FIG. 3b, the records are shown sequentially according to an id value of their question id fields 21a. Those skilled in the art would recognize that the records may be organized in another logical order or any relational manner. Records 21 include the question id field 21a, which is a unique identification number for uniquely identifying each record in the database 11a. All of the questions in the database 11a are grouped into question sets 22. These sets include biographical, gender, life style question sets 22a-22c, and etc. For example, the biographical question set 22a includes questions regarding the participant's biography, such as the question of the record 21 having the id value 102. A text of the question, placed in a question text field 21c, inquires the participant's birth date by requesting: “Please enter the month of your birth date.” The gender question set 22b includes an inquiry of the gender of the participant and the life style question set 22c includes inquiries about the participant's life style.

The inventive system interactively guides the participant to exhaustively capture certain personal biographical, lifestyle, medical history, and any other information provided by the participant. The questions database 11a may be specifically set up or populated for various uses to which the invention may be applied. Each such use may include many different question sets 22 utilized for solicitation of different information including for different gradients or refinements of such information. The database 11a includes the question sets 22 comprising questions particularly prepared to further elicit pertinent information. For example, in one illustrative embodiment describing a medical application of the invention, the questions database 11a includes the question sets 22, listed in Table 1. In such case, the question-sets include core questions developed according to standard clinically validated instruments, or derived from diagnostic criteria in the Diagnostic and Statistical Manual of Mental Disorders (DSM-IV) of the American Psychiatric Association. Furthermore, if a particular overseer wishes to modify any of the question sets 22, the questions identified in the question id field 21a as well as the make up of the question sets 22 can be easily customized. This may be achieved by rewriting the text in the question text field 21c and/or rewriting the set code in the question set code field 21b.

TABLE 1

| SCREENER | EXPLORER | TRACKER |
|---|---|---|
| QUESTIONS SETS | QUESTIONS SETS | QUESTIONS SETS |
| Biographical | | |
| Gender | | |
| Life Style | | |
| Life Data | | |
| Trauma | | |
| Medical History | | |
| Family psychiatric | | |
| Psychiatric | | |
| Alcohol | Alcohol | Alcohol |
| Drug | Drug | Drug |
| Eating | Eating | Eating |
| Anxiety | Anxiety | Anxiety |
| Mood | Mood | Mood |
| Stress | | Stress |
| | | General |
| | | Universal |

The question sets 22 are listed in a Screener column of Table 1. The questions in these question sets 22 are designed to elicit in-depth information about the participant's susceptibility to various syndromes and to determine which problem areas may require further exploration. Depending on the participant's replies to the question sets in the Screener column, the inventive system then guides the participant into a follow-up level of the question sets listed in the column of Table 1 entitled Explorer. The question sets in the Explorer column are designed to capture more detailed information in the specific problem areas indicated. The Explorer column lists the question sets that are designed to further explore a particular condition or a specific problem if such condition was detected or discovered by the questions in the Screener section.

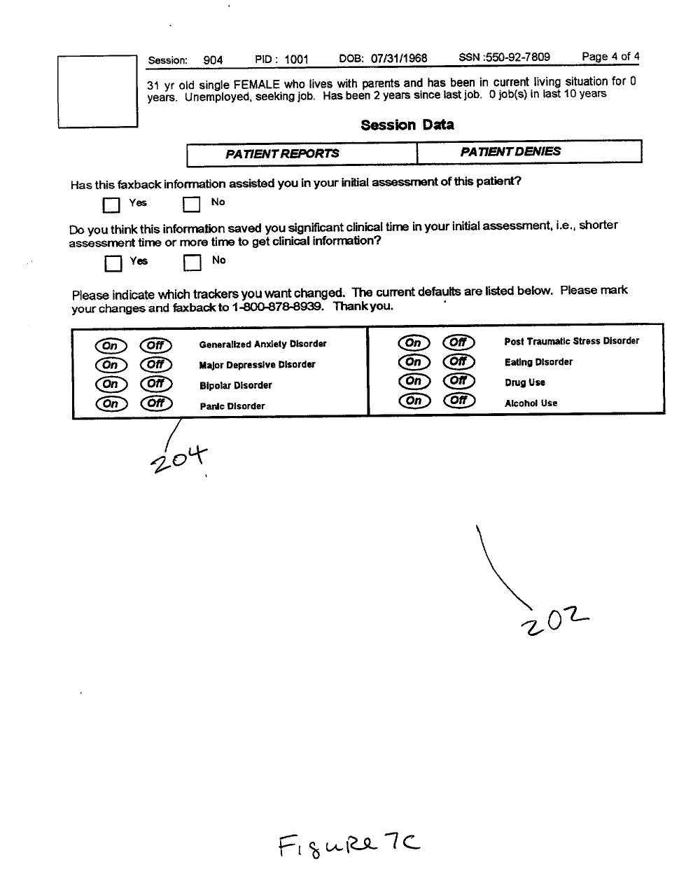

Finally once the participant has replied to the questions in the relevant question sets of the Screener and Explorer columns, the replies are stored and analyzed. Additionally, the participant's profile 200 (FIGS. 7a and 7b) is created from the reply data captured in the answers database 11b, as will be described below. This profile is then automatically forwarded to the overseer, e.g., a treating physician, for review in advance of the in-person meeting between the participant and the overseer. The overseer can annotate the report 202 (FIG. 7c) and send it back to the inbound system 12c, where the annotations are captured and stored in the answers database 11b. Based on this additional information 204 (FIG. 7c), the participant will be asked to reply to the question set 22 selected by the overseer, such as those listed in the Tracker column of Table 1. These question sets are designed to track conditions discovered and explored by prior questioning as well as to solicit any follow-up and new development information during later sessions.

A screener list of question sets may be selected solely by the computer, particularly where information gathering is in a specific field, such as the medical field here. Subsequent lists of questions sets implementing exploring and/or tracking functions are preferably implemented with the participation of an overseer.

The questions belonging to the particular question sets 22 shown in Table 1 are distinguished and identified by the set code stored in the set code field 21b. Moreover, specific functions and any special processing identified by the set code field 21b may be attached to the individual question sets 22. In other words, each question set 22, identified by codes or names such as those shown in Table 1, may have special processing instructions or actions to be performed associated with it. An example of such special processing is to alert the overseer, e.g., call a doctor or the doctor's beeper, when a pre-defined reply is given to a specific question.

In the illustrative embodiment being described, the actual text of the question to be asked is included within each record 21 in the question text field 21c. Other embodiments comprise the text in separate files or other addressed database records of various data types, including text, hypertext markup language (HTML), audio, or video files. One such embodiment will be further described below with reference to FIG. 3d.

A speech identification field 21d is used for indicating the order of the questions within each question set 22. A prompt text field 21e comprises text to give direction to the participant on how to reply to the question in the question text field 21c. Other embodiments for implementing this field are also envisioned. In these envisioned embodiments, similar to the text field 21c, the prompt text may be a separate file or record with data of various types including text, HTML, audio, and video. For example, in situations where the questions are being asked via the IVR system, after the question is posed, the participant may be told to enter three digits, seven or a some other combination of digits as prescribed in a reply text field 21e, e.g., “enter nine digits.” If the participant hesitates or enters the wrong number of digits, the participant will be reminded by playback of the same text in the prompt field 21e, e.g., “enter nine digits.”

A prompt speech id field 21f identifies a particular prompt text when the text is not included within the record 21. A short description field 21g holds the shorthand description of the question text in the field 21c. For example, the question in the record of the biographical question set 22a with the id field 21a value 105 is identified as the social security number (SSN).

A report sequence field 21h is used to order the information in a report, which is prepared from analyzed replies to questions in records 21. A reply type field 21i is used to identify the type of valid replies allowed. For example, when IVR is used, possible values may be assigned by digits 0-9. These values are shown in Table 2. A maximum keys field 21j identifies the maximum number characters that are accepted by the system. The maximum keys field 21j may also indicate a minimum and maximum range of input that is acceptable in response to the asked questions. The maximum keys field 21j may be used to verify information accepted from the IVR enabled system, e-mail, fax, or data entry through the Internet browsers.

TABLE 2

0 = Open reply type, any value may be accepted
2 = Allow input of a “1” or “2”
3 = Allow input of a “1”, “2”, or “3”
4 = Allow input of a “1”, “2”, “3”, or “4”
5 = Allow input of a “1”, “2”, “3”, “4”, or “5”
6 = Allow input of a “1”, “2”, “3”, “4”, “5”, or “6”
7 = Allow input of a “1”, “2”, “3”, “4”, “5”, “6”, or “7”
8 = Allow input of a “1”, “2”, “3”, “4”, “5”, “6”, “7”, or “8”
9 = Allow input of a “1”, “2”, “3”, “4”, “5”, “6”, “7”, “8”, or “9”

A reply open field 21k identifies the id value, i.e., stored in the question id field 21a, of the next question to be asked or presented to the participant. If the value of the reply open field 21k is missing or is a predetermined value such as zero, then the branching value is located in reply fields 211. The reply fields 211 identifies the id value of the next question to be asked or presented to the participant. These follow-up questions depend on the participant's replies. For example, if the reply to the question from the gender question set 22b having the question id field 21a value of 200 is “1”, i.e., male, the follow-up will be the question with the id value of 204, thereby skipping a question intended for women only. If the reply to the gender question with the id value 200 is “2”, i.e., female, then the follow-up id value 201 is retrieved from the second reply field 211.

The remaining fields 21m identify the person who created the record, the person who most recently update the record 21 and the date of creation of the record and the date of the update. Fields 21m also identify the effective and expiration dates of the records 21.

The questions database 11a of the present invention is able to make logical decision branches or jumps based on any controlled input provided by the participant. All potential input is accounted for and proper branching instructions are provided. The questions database 11a is easily manipulated to change these logical branching connections without modifying the text of the questions by changing the values in the reply fields 21k and 211 used for decision branching. To better explain this feature, reference is made to FIG. 3c, which shows logical navigation through the question sets 22a-22e shown in FIG. 3b. These question sets 22 were selected because they represent varying navigational patterns. All of the questions in the biographical screener question set 22a are informational. Decisions are not necessarily made in response to the received replies. Therefore, all branching is performed in a so-called single threaded or fall-through fashion. For example, a question BiS.0 is asked of the participant and the reply is successfully accepted. A following question to be asked will be question BiS.1. The id value of the question BiS.1 is retrieved from the reply open field 21k (FIG. 3b) of the question BiS.0. Similarly, a question BiS2, will be the next question.

The gender screener question set 22b performs both fall-through and branching logic. In such case, after a GeS.0 question (question id 21a value 200) is asked, requesting the sex of the participant, only two values are accepted. The value held in the reply type field 21i (FIG. 3b) of that record indicates that the branching instruction is a function of the user reply, not from the reply open field 21k (FIG. 3b). If the reply to the question GeS.0 is “1” or male, then the next question asked is question GeS.4. The reference to question GeS.4 (question id 21a value 204) is retrieved from the reply1 reply field 211 (FIG. 3b). If, on the other hand, the reply to the question GeS.0 is “2” or female, the next question asked is GeS.1 (question id 21a value 201). The reference to question GeS.0 is retrieved from the reply2 reply field 211 (FIG. 3b).

The life style screener question set 22c mostly follows the branching logic. For example, there are five possible replies to question LSS.2 (question id 21 value 1107). The branching instructions are retrieved from the corresponding reply fields 211 (FIG. 3b) 1-5. The trauma screener question set 22d is an example of the quick sorting out of unnecessary facts and immediate focus on the particular needs of the participant in response to the received replies. By asking directed questions, the participant is navigated into three different medical screener question sets MEDSCR1, MEDSCR2, and MEDSCR3. These question sets may deal with issues particular to the needs of the participant. For example MEDSCR1 includes questions regarding the head injuries complained of by the participant, MEDSCR2 includes questions regarding internal poisoning, and MEDSCR3 includes questions regarding skin disorders the participant might mention.

As mentioned above, FIG. 3d presents an expanded layout of a record 23 for the questions database 11a (FIG. 1a). Two additional fields are provided including a post function field 23p that indicates a function or procedure to be initiated after a noteworthy reply is received from the participant. In the design described with reference to FIG. 3a these functions are placed in a table referenced by the set code value stored in the question set code 21b (FIG. 3a). The post function 23p may return an alternative question routing overriding the routing determined by reply fields 23k and 231. Another additional field is error id field 23o that identifies a characteristic value in the event the function called from the field 23p returns an error. In such case the reply fields 23k and 231 are assigned values of particular questions to be asked or to prompt the participant in an error handling route branching.

Furthermore, an auxiliary content table 24 is linked to the record 23, as shown in FIG. 3d, to define more than one type of question associated with each record 21 (FIG. 3b). Table 24 includes the following fields:

1. a primary key question id field 24a to identify the related questions record 23;

2. a sequence number field 24b to hold a unique number assigned to the auxiliary content table 24;

3. a speech type field 24c to identify the type of speech element defined by the record, e.g., text, audio, and video;

4. a speech file field 24d and speech index field 24e for holding the name or pointer to recorded audio elements to be retrieved and played;

5. a system phrase, system data 1, and system data 2 fields 24f to hold the name or pointer to recorded special system audio elements to be retrieved and played; and

6. a lookup function field 24g used to define an application program interface API callout that may invoke additional audio elements at overseer's command. For example the auxiliary content table 24 may be a prerecorded audio file, a special system audio element such as current date/time. Additionally, a lookup function 24g may be invoked for returning an audio element as well as for performing application API activity such as database inquiries, additions, modifications, and deletions.

“Answers” Database

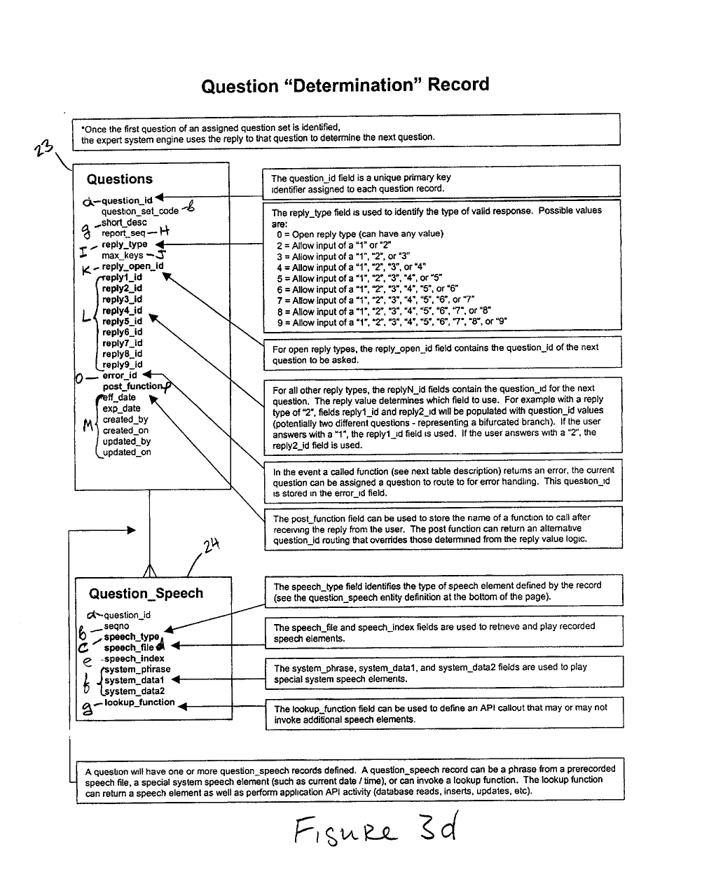

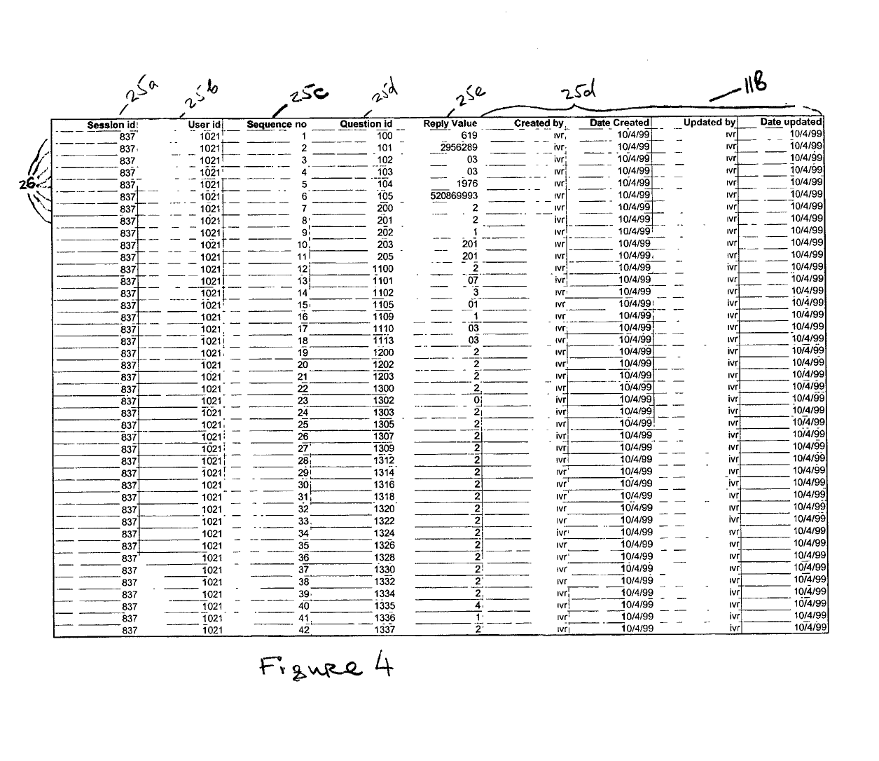

All replies to the questions that are posed to the participants and received from the participants as described above are recorded in the answers database 11b (FIG. 1a). A record layout and an example of the answers database 11b is shown in FIG. 4. Records 26 are shown sequentially, in ascending order, indexed by a sequence value in the sequence number field 25c. This is done to make the description of the invention flow more naturally. However those skilled in the art will appreciate that these records may be organized in any logical, sequential, and relational order, and may be intermixed with records of other origins and record formats including those of the questions database 11a (FIG. 1a).

Each record 26 of the answers database 11b includes the following fields:

1) A session id field 25a for identifying a particular session or conversation when the replies were provided by a particular participant. In the present example, all replies were collected during session 837.

2) A participant id field 25b for identifying the particular participant providing the reply. In the present example, all replies were collected from the participant identified by number 1021.

3) A question id field 25d used for identifying the question being responded to. Field 25dcorresponds to the question id field 21 a of FIG. 3b.

4) A reply value field 25e used for recording the actual value of the provided replies. This field may be substituted by a pointer to another record or a file that would actually hold the reply if the size of the reply is too large. Such may occur if the reply is an audio or other multimedia recording.

5) A method reply value fields 25d for identifying the type of method used to capture and update the reply value as well as the date and the time of such capture and update.

Data Collection

As mentioned above, the present invention in one of its illustrative embodiments may be used for information gathering and analysis in psychiatric, dental, and other medical practices by private doctors, clinics, and hospitals. As shown in FIG. 5, the participant may initiate the first contact by calling the doctor's office using devices 14 (FIG. 1a), mailing an inquiry letter, or establishing a contact with the doctor's office in some other manner, including a visit, all as represented in step 70. Here, the list of questions sets is determined without the aid of an overseer since there has been no previous contact on which to base a selection of questions sets.

In step 72, a screener in the doctor's office determines if the caller is a new authorized participant and then will assign and provide to the participant a unique participant id and a telephone number to call or the Internet address of a website to access. The participant is then asked to call the provided number or visit the indicated website, e.g., using the Internet browsing programs. The screener function may be automated and the question sets to be asked and the participant provided replies may be created in a manner similar to those described with reference to the previously discussed databases 11a and 11b. Questions asked by the screener, automated or human, may include questions regarding the participant's insurance, the validity of which may then be checked by accessing databases 17. The participant's prior medical history is retrieved from the databases 17. The accessing of the databases 17 and retrieving of information records, e.g., medical history, may be performed by common programming techniques which may use protocol and format conversion, including the use of the Internet browsing programs.

The list of question sets implementing the screener function may be selected with or without the participation of the overseer, depending on circumstances.

In step 74, the overseer for the screening and the participant may agree on a schedule for an appointment for the participant to see the doctor. (The doctor may subsequently assume the functions of overseer, or the doctor may share those functions with the overseer for the screening.) The schedule information is stored in the database 11b together with the participant's insurance company name, identification and other participant information. The participant may then be connected to the computing device 12 (FIG. 1a), or as indicated in step 76, call the number previously provided in step 72 at a later time prior to the scheduled appointment. In step 78, the questions from the questions database 11a are selected and presented to the participant. The participant's replies to the questions are saved by the explorer functions of the present invention in the answers database 11b. Any additional information regarding the participant entered in step 80 is also saved.

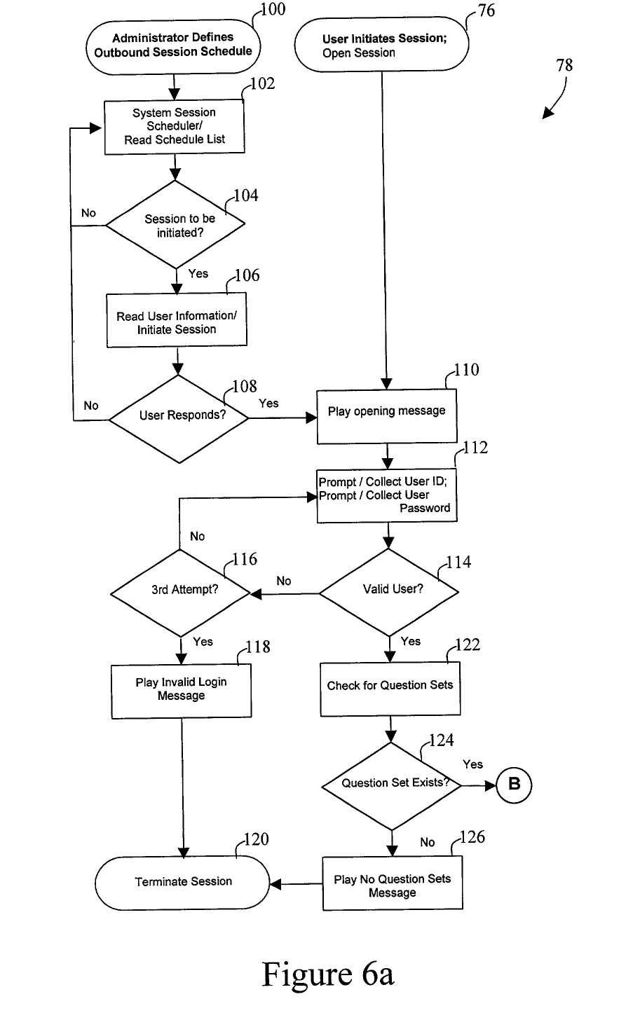

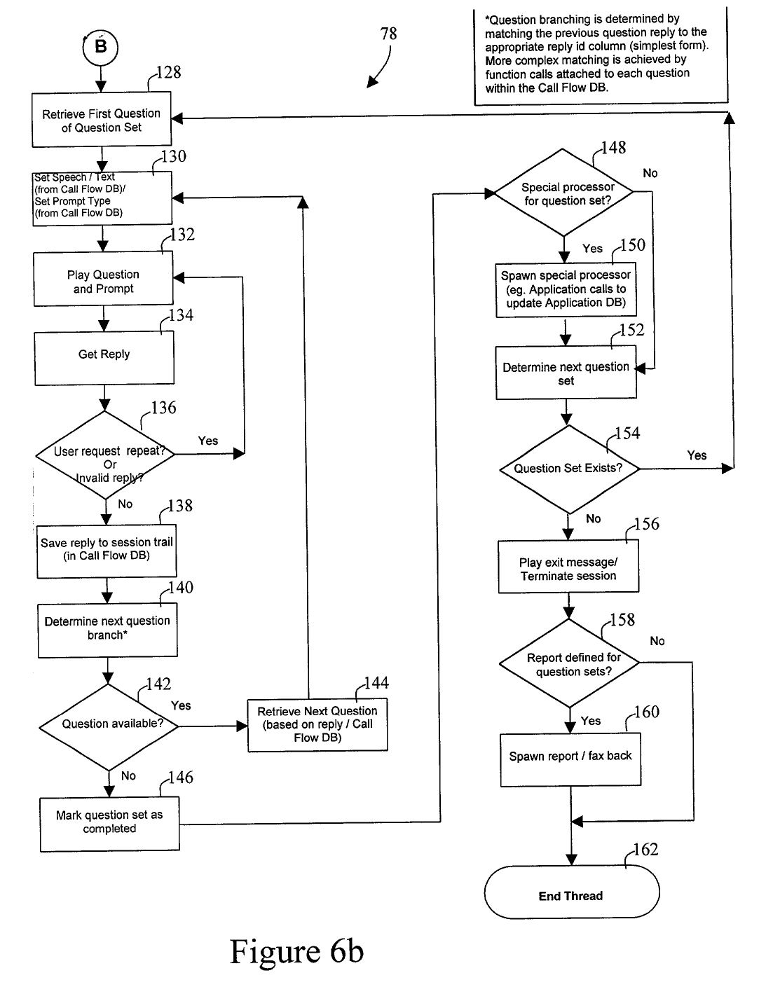

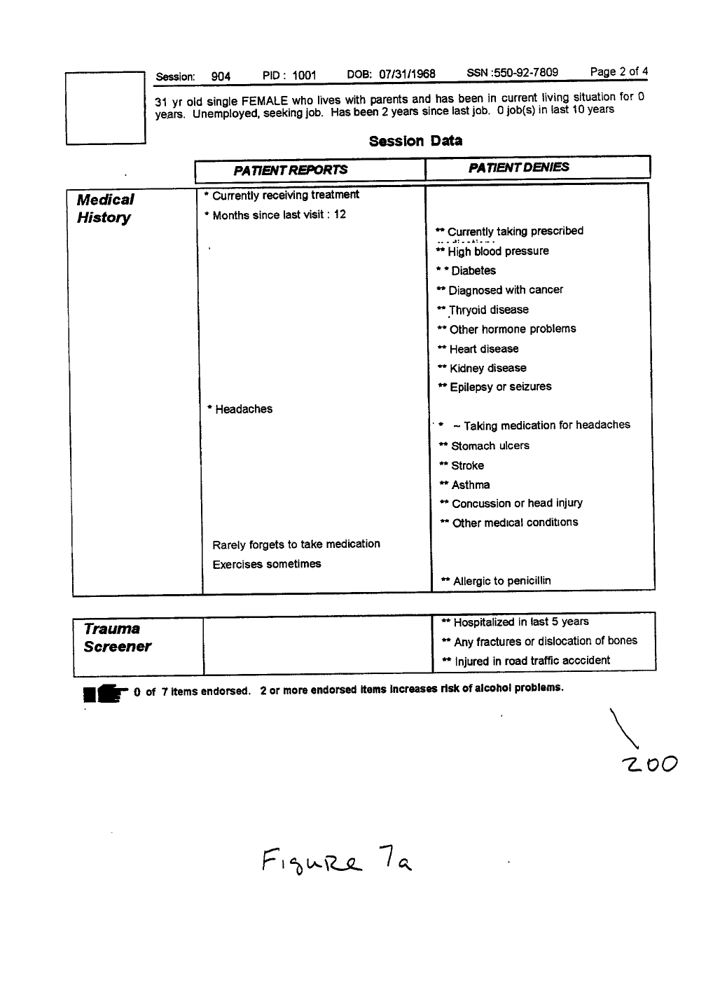

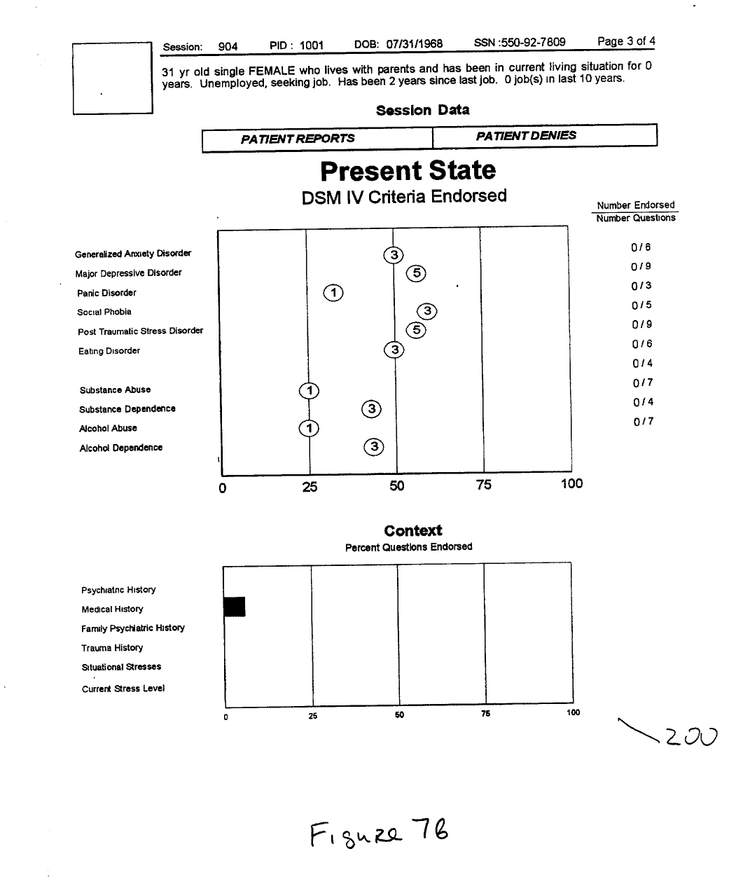

To better explain the functionality of the explorer functions of step 78, reference is made to FIG. 6. Here, as previously mentioned, the participant initiates a session in step 76. As shown in step 100, a data path from the system to the participant is established. In step 102, a session scheduler reads a schedule list of the participants, which may be stored in any database 11 (FIG. 1a). In step 104, the scheduler determines whether a session with the particular participant should be initiated. If not, the scheduler returns to step 102 to choose another participant. If the determination in step 104 is positive, then in step 106 the explorer functions 78 retrieve the participant's personal information from database 11b or, alternatively, if such data has not yet been provided, retrieves the necessary information from the databases 17 (FIG. 5).

In step 106, the establishment of a data path to the participant is further attempted. In step 108, it is determined whether the participant has responded. If the participant has not responded, the processing returns to step 102. Alternatively, if the participant has responded by picking up the phone, or by reading the e-mail message and clicking on a particular hyperlink, in step 110 a session opening message, i.e., a greeting, will be played or displayed.

In step 112 the participant is asked to provide his/her unique participant id. In step 114 the provided participant id is tested for validity. Alternatively, the participant's voice signature may be captured, e.g., in a file, when an audio reply is provided in response to a question during the screening process. Such voice signature may be used to correctly identify the participant. Other biometrics may also be used for participant identification as well as for information collection purposes. Analysis of the provided audio replies can be performed by comparing the stored voice file to newly collected sounds and the information may be extracted to identify stress and speech slurring.

If it is determined that the participant id is not valid the participant is asked to re-enter the participant id in step 112. If after a pre-determined number of attempts the participant fails to provide a valid participant id, as determined in step 116, a message indicating that the login was invalid is played in step 118 and in step 120 the session is terminated. If the participant identification is valid, as determined in step 114, in step 122, the explorer functions 78 determine the appropriate question set to be asked of the identified participant. The participant id determines the questions to be asked. If there is no appropriate question set, as determined in step 124, e.g., the appropriate question set already has been used, the participant, in step 126, is informed that there are no questions to be asked and the session is terminated in step 120. When an appropriate question set is identified in step 128 (FIG. 6b), the first question of that set is retrieved. In step 130, the type of interaction is determined, e.g., speech or via the Internet through written text. In step 132 the question is asked along with an instructional prompt identifying how the participant is to respond.

In step 134, the explorer functions 78 waits or stays idle until the participant replies are entered and accepted. If the participant hesitates too long or an invalid reply is received, as determined in step 136, the question is repeated in step 132. If, the reply is accepted, it is saved in the answers database 11b (FIG. 5) in step 138. As previously mentioned, the replies accepted by the present invention, are not limited to IVR, Voice Recognition, Teletype Device is (TTD) and computer generated replies.

The next question branch is determined in step 140. If a branch is available, as determined in step 142, the follow-up question is retrieved in step 144 and the processing of the explorer functions 78 is resumed in step 130. If there are no more questions available, a particular question set 22 (FIG. 3b) is marked as completed for this particular participant in step 146.

The questions may be selected in a “non-linear” manner. The selection of the current (or Nth) question is based on some or all of the participant response data and not necessarily by the answer to the previous (Nth−1) question. Thus, feedback mechanisms are employed beyond simple branching structures coded into the question sequence, which look only at the answer to the previous question. Instead, questions are selected based on the range of participant replies including:

1) input selections from one and more sessions;

2) participant response accuracy;

3) participant voice characteristics;

4) participant delay in answering the call or responding to a question;

5) the extent to which the participant completes the session;

6) participant responses which do not match previously detected response patterns;

7) participant response times and other behaviors; and

8) information from other participant sessions.

The determination of the Nth question may be represented by a function where the range of replies described above is denoted as variables (R₁, R₂, . . . R_{N-1}). Moreover, evaluation of such function may provide different weights to each of the variables Rᵢ to scale, increase or decrease their importance or to eliminate each previous reply datum. For example, the alcohol explorer question set is activated when the participant provides affirmative replies to the question “Have you been admitted to the emergency room one or more times in the past 6 months?” and the follow-up question “Have you ever tried to cut down on your drinking?” Additionally, any unusual behavior such as a delay in responding to the follow-up question or any inconsistency with other previous replies is taken into account.

The questions can also be selected through parametrically driven feedback mechanisms. Certain parameters are introduced such that when a parameter's threshold is met, other question sets are implemented. For example, when participant response time is extended past set norms, e.g., 30 seconds, a cognitive testing questions set can be added to the question sets already assigned to the participant.

Moreover, the parameter thresholds, e.g., greater than 15% negative change in a global score from base line, may be used to rate the severity of the reply information provided such that the frequency of tracking calls to the participant is increased or the participant is asked to provide replies more often. Automated statistical comparison of the participant's recovery process to a matched sample from the knowledge base 17 (FIG. 1a) of previous participant responses are used to select question sets specifically related to risk of exacerbation of illness. Thus, participants falling behind the normal curve by a threshold percentage would have their frequency of tracking sessions automatically increased.

The selection of questions is dynamic, and at least theoretically, any question in the database may be branched to.

Referring back to FIG. 6b, step 148 determines whether there is a special processor as determined by a table associated with the set description field 21g (FIG. 3a) or with the post function field 23p (FIG. 3d). If the special processor is found, that processor is executed in step 150 and in step 152 the next question set 22 (FIG. 3b) is determined after the special processor is executed or when there is no need for such processor to be executed. If it is determined in step 154 that there is a next question set 22 to be asked, the processing of the explorer functions 78 resume in step 128. If on the other hand, the follow-up question set 22 does not exist, a termination message is sounded or displayed to the participant in step 156. In step 158 the explorer functions 78 determine if a report to the overseer or other authorized person can be produced based on the accepted replies. If it is determined that a report 200 (FIGS. 7a, 7b) can be created, the report 200 (FIGS. 7a, 7b) is produced in step 160 and sent to a predetermined designation on computing devices 20 (FIG. 1a) and facsimile machines 19 (FIG. 1a). If the report 200 (FIGS. 7a, 7b) cannot be created, the explorer functions 78 terminate in step 162.

Referring back to FIG. 5, the participant's session with the explorer functions in step 78 may take place before the appointment scheduled in step 74. After the session, the report 200 (FIGS. 7a, 7b) of the replies provided by the participant is provided to the doctor, the doctor may familiarize himself or herself with the participant's maladies, prior medical history made available by the participant's replies and any other information retrieved from databases 17. The visit of the participant with the doctor will therefore be much more productive because the doctor will be in possession of much more information regarding the participant than was ever possible before the invention. After making assessments of the participant, the doctor may initiate his or her own session with the explorer functions in step 78 providing his or her assessment of the participant in the same manner as described with reference to the explorer functions in FIG. 6. This doctor provided information is stored in the answers database 11b as a separate table or records 26 (FIG. 4) having a key field for linking such information to a specific participant session similar to the session id field 25a (FIG. 4). Alternatively, such information may simply be added to the existing answer record 26 (FIG. 4) of a particular participant.

Based on this additional overseer provided information, information provided by the participant, and any external information related to the participant retrieved from database 17, the explorer functions in step 78 may produce additional reports 200 (FIGS. 7a, 7b) and suggestions for follow-up treatment that may have been overlooked without all the information being available. Based on this collected and analyzed information and on the information collected and analyzed from the participants suffering from similar maladies, in step 84, the doctor may prescribe a treatment for the participant. The prescribed treatments may include drug therapies, physical therapies, or other activities. While following the doctor prescribed therapies the participant will participate in the follow-up sessions with the tracker functions of the invention as shown in step 88. The tracker functions accept the participant information, analyze it with reference to the previously accepted and stored information, doctor provided information, and any external information retrieved from database 17 and creates reports 200 (FIGS. 7a, 7b), which it then forwards to predetermined recipients as described above.

The tracker functions are similar to the explorer functions described with reference to FIGS. 6a and 6b. The tracker functions interactively elicit, by posing the so-called tracker question sets 22 (FIG. 3b), and capture information from the participant during the course of treatment and enable the treating physician to monitor the progress of such treatment and adjust that treatment according to the information elicited. The tracker question sets 22 (FIG. 3b) may be posed by telephone calls or communication via the Internet automatically initiated by the inventive system itself at clinically-appropriate intervals specified by the treating physician, with due allowance being made for automatic, repeated calls until a patient has been reached and the necessary information elicited. Additionally, tracker question sets 22 (FIG. 3b) offer an educational/self-treatment component where patients can research their symptoms, initially through repetition, and later via disease-specific instructional modules.

In step 92, the doctor, analyzing tracker reports, may then suggest for the participant to come back to the doctor's office for a repeat visit as shown in step 80. Alternatively, the doctor may suggest repeat follow-up sessions as shown in step 86. Those follow-up sessions may include questions that are the same or different from the ones prescribed before. Those differences are determined by the doctor and adjusted in the database 11a by changing the special processor fields 21o (FIG. 3a) and 23p (FIG. 3d), which are queried by both the explorer functions in step 78 and the tracker functions in step 88 as shown in step 150 of the tracker functions (FIG. 6b).

As described above, after the replies to the question sets 22 (FIG. 3b) are collected, the analysis or findings are reported to an authorized person at a predetermined location via the faxback feature of the present invention, the e-mail, regular mail, audio phone message, at a predefined Internet website and other electronic methods.

Knowledge Base

The participant replies are captured and re-used together with other information sedimented by the day-to-day operation into the answers database 11b (FIG. 1a). Access to such information may then be provided by the overseer to health care organizations, government agencies, pharmaceutical companies, employers, and others for epidemiological, economic and other purposes. The information, e.g., epidemiological information, gathered by the inventive system may be utilized while the individual participants will remain anonymous. It is planned to aggregate such data in a knowledge base, building a depository of patient demographics, treatments, and outcomes data. From such knowledge base, a statistical model of normative replies to various treatment protocols are created. This should permit on-the-fly statistical comparisons to the normative curve of the individual participant replies, allowing the medical practitioner to evaluate whether the participant is getting well as fast as he or she should in comparison to similar participants. That, together with the trackers question sets described above, will in turn permit mid-course corrections in the physician's approach to treatment, rather than relegating him or her to outcomes research that by definition yields some of the most outcomes-critical information only long after the fact.

This unique capability allows for dynamic knowledge base sampling over time, as compared to typical static outcome data, at low to zero-cost of data collection. Healthcare providers and pharmaceutical companies are thus empowered to continuously examine and improve courses of treatment by extrapolating relevant data, and comparing the individual replies against matched population groups. It is planned that the knowledge base be included as part of the databases 17 (FIGS. 1a and 5) to be queried and utilized for the report 200 (FIGS. 7a, 7b) creation.

Uses

Patient and Treatment Information

The enormous need for patient self assessment and treatment information may be satisfied by the invention in virtually all entities within the health care industry including health care provider groups; managed care organizations; physician practice management companies; independent health care provider/group practices; and pharmaceutical companies. The patient self-assessment and treatment information collected by the invention may benefit these entities in the following areas:

1) New Case Identification.

a) Raising levels of efficiency, accuracy, consistency and effectiveness of the health care provider, while lowering costs of initial screening process.

b) Increasing productivity and value for the first in-person consultation with a physician, therapist, or a psychiatrist.

c) Generating individual baseline and aggregate knowledge base for patient/illness profiling.

d) Allowing for covert screening with adjustable sensitivity.

e) Enhances the participant's comfort level for answering sensitive questions because the replies are given in privacy.

2) Clinical trials.

a) Raising levels of efficiency, accuracy, consistency and effectiveness of the health care provider in obtaining the reply data.

b) Providing a consistent knowledge base in ready form for analysis.

c) Enabling testing against normative data, rather than just placebo. Eliminating need for physical visits and/or repeat phone calls to provide feedback.

d) Accelerating approval process and time to market for new drugs.

3) Satisfaction Surveys.

a) Promoting more candid and widespread participation resulting in quicker response to feedback.

b) Compiling a knowledge base of feedback and allowing comparison of the replies over time.

c) Providing our anonymous outlet to provide feedback.

4) Concurrent Outcomes Analysis.

a) Accessing historical and real-time data.

b) Providing a more robust, flexible, knowledge base for low-to-zero cost.

c) Enhancing treatment through access to historical extrapolated data.

d) Providing access to objective data on treatment replies.

e) Reducing time on treatments that are not effective

5) Case Management/Decision Support by

a) Improving treatment compliance.

b) Providing for in-situ clinical course correction.

c) Facilitating proactive and reliable communication of high-value actionable information to physician.

Telediagnosis/Telemonitoring

The inventive system may be effectively used in telemonitoring, for example, in the home care industry where monitoring the elderly and home bound patients according to the inventive method will enable continuous record to be kept on individual patients and alerts to be issued to overseer when changes arise. For example, if a participant was consistently giving a reply “good” to the question “how do you feel” and then on one occasion the reply received is “bad,” the inventive method may immediately alert an overseer to that fact after comparing all the answers and detecting a change. In one embodiment of the present invention where audio replies are recorded and stored, together with the reply content, i.e., “good” and “bad,” the voice characteristics may be compared to detect stress, etc.

Additionally, the invention may be used to provide a prescription drug reminder, verify compliance, and accept a prescription drug refill request. The invention may further be used as a patient appointment reminder, but is of course not limited to any context described above and has extremely widespread application.

Among the areas of obvious application is the area of behavioral medicine/psychiatry including the treatment of the various addictions. Telemonitoring can be especially fruitful in this area, and can be coupled as well with the remote application of diagnostic instruments to provide a dynamic tracking system for outpatients undergoing treatment for psychiatric/behavioral problems and the treatment of addictions.

There are a number of reasons why psychiatric/behavioral medical services represent a fertile area for the application of the present method. Among them are the relatively high number of routine verbal transactions designed to gather medical data or deliver treatment; the existence of a large complement of reliable and valid self-report or interviewer-rated assessment instruments to gather data and do analyses of treatment compliance and efficacy; and obtain reliable verbal reports of the participant's symptoms and the efficacy of treatment which could dramatically cut costs and support the efficient delivery and tracking of services,

Other uses in Medical Care

The inventive system can fill gaps in medical markets beyond behavioral healthcare. In surgical care the tracker feature of the present invention may be used for post-operative fever and pain monitoring as well as wound healing. The medication reminder feature may be used to help maintain antibiotic therapy. In obstetrics and gynecology, the screener/explorer feature may be used for high-risk factor assessment, the tracker feature in post-delivery, e.g., pain or fever monitoring and the medication reminder feature for pre-natal vitamins, diet, prohibition reminders, etc. In general medical/primary care practice, the screener/explorer feature may be used for standardized function assessment and psychiatric evaluation, the tracker feature in pain management, and the medication reminder feature for chronic care settings such as homecare and most chronic diseases, e.g., hypertension, Crohn's disease and ulcerative colitis, or cognitive impairment in geriatric patients.

Claims Processing

The inventive system enables health care service providers to submit insurance reimbursement claims to a central payer, such as Medicaid. The claim forms or claim reports are created with the use of the participant provided information and the diagnosis information provided by a treating physician retrieved from the answers database 11a (FIG. 1a) and from databases 17. The claim reports may use standard medical reimbursement forms and forward these reports via the outbound system 12d (FIG. 1b) which utilizes generally available e-mail and fax technology without the need to install expensive or complex equipment, and at a fraction of the cost of current methods of claims submission involving hard copy and the mails. Furthermore, the digital nature of the information being reported allows for such transmitted information being captured directly into an audit database and re-used for audit and actuarial purposes.

Moreover, since most medical offices already have access to e-mail using computing devices 20 (FIG. 1a) and/or fax machines 19 (FIG. 1a), no overhead for complex and expensive computer equipment need be incurred. The service providers can avoid the costs of upgrading or installing traditional EDI-enabled computer equipment. Doctors can control the information they are submitting by e-mail and fax and are able to submit claims more quickly and easily than by mailing them. Because the present invention enables payers to eliminate overhead on a dramatic scale and to process claims much more efficiently, service providers save the costs of financing accounts receivable. In addition, with the e-mail and fax-enabled transmissions, unacceptable claims are rejected much more quickly then the traditional mail in submissions. For example, it has been estimated that from 12-20% of claims submitted are submitted to the wrong insurer. Typically these erroneously submitted claims are not detected until they have already cost the insurer more than $10 a claim in processing charges.

In alternative embodiments, the invention may be utilized for information gathering i.e., to capture particular data, and for issuance of insurance reimbursement claims for life, auto, home and other insurance policies, e.g., Worker's Compensation Incident Reporting First Report of Injury (FROI).

Advertisements

In one embodiment, the invention may be implemented to capture the replies to advertisements, such as help wanted advertisements, in newspapers, and other media, or responses to requests for proposals (RFP) in contract evaluations. This is achieved through the IVR and/or the Internet by interactively questioning the participant, i.e., the persons responding to such ads, using specifically designed questions from the database 11a (FIG. 1a). These questions may include inquiries as to the participant qualifications in the case of the help wanted ads or queries for other information in other cases. The replies to these inquiries are stored in the database 11b (FIG. 1a). The captured information is sorted according to criteria selected by the overseer, i.e., a company or a client who has placed the ad. This embodiment of the invention may forgo the screening step 72 (FIG. 5) and provide the directions within the ad on how to engage the inventive system by providing the telephone number of the dial-up IVR or the Internet address.

Reports may then be provided to the client in a fax or electronic form comprising the analysis of the applicants sorted according to any particular preferential trait predetermined by the overseer. For example, respondents may be ranked in such reports on the basis of their replies to particular questions or their possession of desired qualifications. As a consequence, the tedious and expensive process of sorting and ranking the resumes can be automated and the results provided to the client in a concise ranked and indexed summary or other form. In addition, all the information received may be compiled in a knowledge base described above, for later use in filling opening job positions as they occur as well as for statistical and other purposes.

Self Improvement

In yet another embodiment of the present invention the questions database 11b (FIG. 1a) may include questions designed to assess the knowledge of the participant in certain disciplines, for example SAT, LSAT, college entrance, bar exams and any other examination preparatory and accreditation courses. The students are automatically guided to appropriate question sets 22 (FIG. 3b) to provide appropriate level of questions and feedback. The questions may be prepared to gauge a participants' knowledge in foreign languages, math levels, etc. Course enrollment and other administration may be easily addressed by the inventive method.

Inventory Control

In additional embodiments, the invention may be utilized for inventory control e.g., in a video rental store where the participant replies with a movie title, movie director or some character name to reserve a movie. By referring to auxiliary databases 17 possessing relevant movie information the invention determines the requested movie. In this embodiment, the knowledge base of participant preferences may be used to offer personalized selections to the participants. Such knowledge bases may be used for consumer product marketing, market surveys, to increase product awareness, and to distribute product information using the outbound system 12d (FIG. 1d).

The present invention may be further used to accommodate product recalls and customer service, to research and purchase stocks, to make take out orders at restaurants and stores, and many other applications.

While the invention has been particularly shown and described with respect to illustrative and preferred embodiments thereof, it will be understood by those skilled in the art that the foregoing and other changes in form and details may be made therein without departing from the spirit and scope of the invention that should be limited only by the scope of the appended claims.

1. A method of assembling information for describing a condition of each of a plurality of participants, said method comprising the steps of:
adjusting a questions list comprising a plurality of questions assembled with the participation of at least one overseer for each of said plurality of participants; and
accepting one or more replies in response to each questions list provided by each of said plurality of participants.

2. The method of claim 1, comprising a step of creating said plurality of questions for each list, each of said plurality of questions intended to elicit one or more replies providing information for describing one or more aspects of the condition.

3. The method of claim 2, comprising a step of creating one or more questions sets, each said one or more questions sets comprising said plurality of questions.

4. The method of claim 3, comprising a step of creating said questions list for each of said plurality of participants, said questions list comprising said one or more questions sets.

5. The method of claim 4, comprising a step of establishing at least one participant data path for communicating at least one questions list to at least one participant and for accepting said one or more replies from said at least one participant.

6. The method of claim 5, further comprising a step of establishing at least one overseer data path for communicating at least one report to at least one overseer and for accepting one or more directives from said at least one overseer.

7. The method of claim 6, further comprising a step of communicating said at least one questions list to said at least one participant via said at least one participant data path.

8. The method of claim 7, further comprising a step of creating said at least one report, said at least one report comprising an analysis of said one or more replies from said at least one patent and said one or more directives from said at least one overseer.

9. The method of claim 8, further comprising a step of communicating said at least one report to said at least one overseer via said at least one overseer data path.

10. The method of claim 9, further comprising a step of accumulating replies and directives into a knowledge base.

11. A computer program device readable by a machine, tangibly embodying a program of instructions executable by a machine to perform method steps of assembling information for describing a condition of each of a plurality of participants, said method comprising the steps of:
adjusting a questions list comprising a plurality of questions assembled with the participation of at least one overseer for each of said plurality of participants; and
accepting one or more replies in response to each questions list provided by each of said plurality of participants.

12. A method of collecting a plurality of replies from at least one participant into a first database residing on one or more computing devices, the one or more replies describing a condition of the at least one participant provided in response to a plurality of questions, said plurality of questions being selected by at least one overseer, the at least one overseer providing directions, said directions directing a selection of the plurality of questions, said method comprising the following steps of:
establishing at least one participant data path for communicating said one or more replies from the at least one participant to the one or more computing devices;
creating one or more question sets, each of said one or more question sets comprising said plurality of questions;
creating at least one question list for each of the at least one participant, said at least one question list comprising said one or more question sets, said one or more question sets comprising said at least one question list are determined from the one or more replies and said one or more overseer responses;
for said plurality of questions in at least one list,
determining a next question for said at least one participant using the one or more replies and one or more directions,
communicating said next question to the at least one participant via said at least one participant data path, and
communicating the one or more replies from the at least one participant via said at least one participant data path.

13. The method of claim 12, further comprising a step of creating said plurality of questions, each of said plurality of questions expected to elicit the one or more replies describing one or more aspects of the condition.

14. The method of claim 13, wherein said at least one participant data path further communicating said plurality of questions from the one or more computing devices to the at least one participant.

15. The method of claim 14, further comprising a step of establishing at least one overseer data path for communicating at least one report from the one or more computing devices to said at least one overseer and for communicating the directives from said at least one overseer to the one or more computing devices.

16. The method of claim 15, further comprising steps of:
creating one or more reports comprising an analysis of the one or more replies and said one or more directives;
communicating said one or more reports to said at least one overseer; and
communicating said one or more directives from said at least one overseer in response to said one or more reports.

17. A method of obtaining information from a person using a computer with the participation of an overseer, comprising the steps of:
providing at least one first set of questions to be presented to the person;
computer generating questions from within each first set and presenting those computer generated questions to the person;
receiving in the computer answers from the person to the questions presented from said at least one first set of questions;
each question presented to the person from said at least one first set being selected by the computer from said at least one first set of questions independently of answers received from the person or in dependence upon at least one answer received from the person;
providing at least one second set of questions to be presented to the person in dependence upon at least one answer received to at least one question presented from at least one first set of questions, said at least one second set of questions being selected through intervention by the overseer;
computer generating questions from said at least one second set of questions and presenting those computer generated questions to the person; and
receiving in the computer answers from the person to questions presented from said at least one second set of questions.

18. The method of claim 17 wherein the step of providing said at least one first set of questions comprises the computer selecting the first question set or sets without overseer participation;

19. The method of claim 17 wherein the step of providing said at least one second set of questions comprises the overseer participating in a selection of at least one second set of questions.

20. A method of obtaining information from a person using a computer with the participation of an overseer, comprising the steps of:
providing a first list of question sets to be presented to the person, each question set comprising at least one question relating to preliminary information or information relating to a condition of the person;
computer generating questions from question sets within the first list and presenting those questions to the person;
receiving in the computer answers from the person to the questions presented from the first list;
providing a second list of question sets to be presented to the person, each question set in the second list comprising at least one question relating to a condition of the person;
computer generating questions from question sets within the second list and presenting those questions to the person;
receiving in the computer answers from the person to the questions presented from the second list;
the overseer participating in providing at least the second list in dependence upon answers received to questions presented from the first list.

21. The method of claim 20 wherein the step of providing said at least one first set of questions comprises the computer selecting said first list of question sets.

22. A method for obtaining information from a person using a computer, comprising the steps of:
from a database of question sets providing at least one set of questions to be presented to the person;
from the database retrieving questions from the first set and presenting those questions to the person;
receiving in the computer answers to questions presented to the person;
the step of retrieving from the database questions from the first set of questions comprising retrieving at least one question in dependence upon an answer to an earlier presented question.

23. A system for obtaining information from a person comprising:
a computer;
a computer readable memory storing questions and in association with the person answers to the questions presented to the person, the memory being accessed by the computer;
the computer being programmed to select any question of a plurality of questions stored in memory to present to a person in dependence upon any answer of a plurality of answers stored in the memory.
